(19) United States
(12) Patent Application Publication
(10) Pub. No.: US 2021/0145620 A1
(43) Pub. Date: May 20, 2021

(54) SYSTEM AND COMPONENTS THEREFOR FOR USE IN TREATING A BONE FRACTURE

(71) Applicant: Pranesh KUMAR, Hamilton (NZ)

(72) Inventor: Pranesh KUMAR, Hamilton (NZ)

(21) Appl. No.: 16/622,450

(22) PCT Filed: Jun. 15, 2018
(86) PCT No.: PCT/NZ2018/050084

§ 371 Date: Dec. 13, 2019

(30) Foreign Application Priority Data
Jun. 15, 2017 (NZ) 730101

(51) Int. Cl. A61F 5/01

(52) U.S. Cl. CPC A61F 5/013; A61F 2005/0167; A61F 2005/0158

(57) ABSTRACT

The specification describes improvements to splints for use in treatment of bone fractures, having particular application for treatment of distal radius fractures. The splints are adjustable, and may be provided in a range of sizes, allowing them to be better customised to a treatment of a patient. In addition, embodiments of the splints are configured for use in treating distal radius fractures treated by surgical fixation.

### TECHNICAL FIELD

The present invention relates to a system and components therefor for use in treating a bone fracture.

### BACKGROUND ART

Bone fractures are a common medical occurrence in which two or more bone fragments are created by a crack or complete break in a bone. Specific conditions are required to promote or optimise healing from such a fracture. For instance, correct alignment of the bone fragments is beneficial to ensure that the body's natural processes repair the fracture with the bones in the unfractured orientation.

Devices such as splints and casts are typically used for extended time periods to assist with achieving correct alignment of the bone. The time required for a splint or cast to be used depends on the person's physiological response, and the severity of the fracture. However, typical time frames for use of a splint or cast range from several weeks to three months or more.

Distal radius factures are a relatively common form of bone fracture. These fractures are defined as being a fracture that occurs within approximately three centimetres from a person's wrist joint. These features are relatively predictable in their location.

Given the frequency with which distal radius fractures occur, and the requirements to promote healing of the fracture, it would be desirable to have a splint which better promotes treatment and healing of the fractures.

Alternatively, it is an objective of the present invention to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

It is an object of the present invention to address the foregoing problems or at least to provide the public with a useful choice.

Further aspects and advantages of the present invention will become apparent from the ensuing description which is given by way of example only.

### DISCLOSURE OF THE INVENTION

According to one aspect of the invention, there is provided a system for use in treating a bone fracture. The system may include a splint as substantially described herein, instructions on how to use the splint, and one or more tools to assist in operating the splint.

According to another aspect of the present invention, there is provided a splint that may be more adaptable to a patient or is more adaptable to a portion of the population.

According to another aspect of the present invention, there is provided a splint which may better fit a patient or a portion of the population.

According to another aspect of the present invention, there is provided a splint that may provide increased patient comfort.

According to another aspect of the present invention, there is a provided a splint which may facilitate, improve or promote repair of bone fractures.

According to another aspect of the present invention, there is provided a splint that may be easier to use.

According to another aspect of the present invention, there is provided a splint that may provide support to bone fragments to improve or promote repair of bone fractures.

According to another aspect of the present invention, there is provided a splint that may improve alignment of bone fragments to facilitate or improve treatment of a bone fracture.

According to another aspect of the present invention, there is provided a splint for use in treating a fracture after surgical fixation of the bone fragments forming the fracture.

In a particularly preferred embodiment, a splint according to the present invention may be structured and/or configured to avoid contact with an area of a patient's body in which a flexor carpi radialis (FCR) incision is made.

In preferred embodiments, the invention described herein is intended to be incorporated into a wrist splint for use in treating a distal radius fracture. For instance, the wrist splint may be that described in PCT Application No. NZ 2014/000117, and that document is incorporated herein in its entirety by reference.

According to another aspect of the present invention, there is provided a splint for use in reducing a fracture which produces a first bone fragment and a second bone fragment, wherein the splint includes

a first restraint portion configured to apply pressure to a volar surface of the patient's forearm and thereby apply pressure to at least one of the first bone fragment and the second bone fragment,
a second restraint portion configured to apply pressure to a dorsal surface of the patient's forearm and thereby to the apply pressure to at least one of the first bone fragment and the second bone fragment,
wherein the orientation of the first restraint portion and the second restraint portion can be adjusted with respect to each other to thereby align the first bone fragment and the second bone fragment in a desired orientation that substantially corresponds to an orientation for the first bone fragment and the second bone fragment in a non-fractured bone, and
a connection assembly between the first restraint portion and the second restraint portion, and wherein
the connection assembly can hold the first restraint portion and the second restraint portion substantially immovably with respect to each other to thereby hold the first bone fragment and the second bone fragment in the desired orientation.

Preferably, the splint may be configured to reduce a fracture in a limb of a patient.

Preferably, the splint is configured to reduce a fracture in an arm of a patient, and more preferably to reduce a distal radial fracture in a patient's arm.

Preferably, the connection assembly includes at least one spacer element and a joint.

Preferably, the at least one spacer element includes a leg having a length.

Preferably, the at least one spacer element further comprises a second leg having a length.

Preferably, at least one of the first leg and the second leg is constructed and/or arranged to be snapped to adjust the length of the leg(s).

Preferably, at least one of the first leg and the second leg includes one or more weakened regions to facilitate the leg(s) being snapped.

Preferably, the splint includes a locking mechanism to prevent movement about the joint.

Preferably, the locking mechanism is a screw.

Preferably, the screw has a non-regular engagement head.

Preferably, the non-regular engagement head is provided by a plurality of apertures.

Preferably, the first restraint portion is substantially rigid.

Preferably, the second restraint portion is substantially rigid.

Preferably the splint further includes a third restraint portion which is secured to the second restraint portion by a joint.

Preferably, the third restraint portion is movably secured to the second restraint portion.

Preferably, the splint includes a locking mechanism to prevent movement about the joint between the third restraint portion and the second restraint portion.

Preferably, the joint between the second restraint portion and the second restraint portion is a ball and socket type joint.

Preferably, the ball and socket type joint comprises a ball component and a socket, and wherein the ball component is releasably engaged in the socket.

Preferably, the ball component is releasably engaged in the socket in a press-fit type connection.

Preferably, the socket includes a first opening configured to enable the ball component to be inserted into the socket.

Preferably, the ball component and the socket have complementary shapes to each other.

Preferably, the ball component is partially spherical and the socket is partially spherical.

Preferably, the ball and socket further comprises an articulating component.

Preferably, the articulating component is configured to be releasably inserted into the socket.

Preferably, the socket includes a second opening to facilitate insertion of the articulating to component into the socket.

Preferably, the articulating component comprises a pair of side walls, and wherein the pair of side walls define a channel to receive the ball component.

Preferably, in-use rotation of the ball component causes the articulating component to rotate.

Preferably, rotation of the ball component brings the partially spherical ball shaped component out of alignment with the partially spherical socket to thereby provide resistance to the ball component being withdrawn from the socket.

Preferably, comprising a locking mechanism to prevent movement at the joint between the second restraint portion and the third restraint portion.

Preferably, the locking mechanism is at least one screw having a non-regular engagement head.

Preferably, the non-regular engagement head is a plurality of apertures.

Preferably, the third restraint portion further comprises an arm.

Preferably, the arm is structured resilient and able to flex in a first orientation.

Preferably, the first orientation allows the patient's wrist to move in the direction between extension and flexion, but wherein the arm provides resistance to said movement.

Preferably, the arm is structured to be substantially inflexible in a second orientation.

Preferably, the second orientation is substantially perpendicular to the first direction.

Preferably, the arm is structured to provide resistance to movement of the wrist between radial deviation and ulnar deviation.

Preferably, the third restraint portion further comprises a loop which is mounted to a distal end of the arm.

Preferably, the loop is releasably mounted to the arm.

Preferably, the arm includes a channel and the loop includes a ball, and wherein the ball is configured to be inserted into the channel to thereby mount the loop to the arm.

Preferably, at least one of the first restraint portion and the second restraint portion include a coating layer on a surface of the restraint portion which in use contacts a surface of the patient's forearm.

Preferably, the coating layer is a foam material.

Preferably, the coating layer is permanently attached to the restraint portion(s).

Preferably, the first restraint portion includes a channel configured to receive the first leg.

Preferably, the first leg includes a plurality of teeth which engage the channel.

Preferably, the first restraint portion includes a channel configured to receive the second leg.

Preferably, the second leg includes a plurality of teeth to engage which engage the channel.

Preferably, at least one of the first leg and the second leg is resilient.

Preferably, at least one of the first leg and the second leg is structured and/or arranged, to bias the teeth to engage the respective channel.

Preferably, at least one of the first leg and the second leg can be bent to cause the respective teeth to disengage the respective channel.

Preferably, the splint is provided in an extra-small, small, medium or large size.

Preferably, the sizes of the first restraint portion are substantially:

| Dimension | Extra-small | | Small | | Medium | | Large | |
|---|---|---|---|---|---|---|---|---|
| A | 98 | mm | 109 | mm | 109 | mm | 109 | mm |
| B | 62.8 | mm | 73.5 | mm | 84.1 | mm | 94.8 | mm |
| C | 12 | mm | 15 | mm | 15 | mm | 15 | mm |

Preferably, the sizes of the second restraint portion are substantially:

| Dimension | Extra-small | Small | | Medium | Large |
|---|---|---|---|---|---|
| A | 74 mm | 79 | mmm | 79 mm | 84 mm |
| B | 65 mm | 75 | mm | 86 mm | 97 mm |
| C | 20 mm | 20 | mm | 19 mm | 19 mm |

Preferably, the splint further includes a tool configured to facilitate fitting of the splint to a patient.

Preferably, the tool includes a body and an extension portion.

Preferably, the tool comprises an engagement portion configured to engage the locking mechanism.

Preferably, the body of the tool includes a channel configured to receive an end of a/the first leg and a/the second leg of the splint to facilitate snap off the first leg and the second leg.

Preferably, the splint further comprises a strap.

Preferably, the strap is attached to the first restraint portion.

Preferably, the strap includes a first end and a second end.

Preferably, the first end of the strap is releasably attached to the first restraint portion.

Preferably, the second end of the strap is releasably attached to the first restraint portion.

Preferably, the strap is releasably attached to the first restraint portion.

Preferably, the strap is attached to a proximal end of the first restraint portion.

Preferably, the first restraint portion further comprises a first loop and a second loop, and wherein the first loop and the second loop are located on side edges of the first restraint portion to each other.

Preferably, the strap extends through the first loop and the second loop.

Preferably, the length of the strap is adjustable.

Preferably, the strap includes a first connector and at least two second connectors, and wherein the first connector can selectively engage one of the two second connectors to thereby releasably attach the first end of the strap to the first connector half and to facilitate adjusting the length of the strap.

Preferably, the first restraint portion is structured to define a clearance zone.

Preferably, the clearance zone is configured to ensure that the first restraint portion does not contact an area of the patient's forearm in which an FCR incision is made.

Preferably, the clearance zone includes a window.

Preferably, the first restraint portion includes a bridge which defines the clearance zone.

Preferably, the bridge is curved.

Other aspects, features and advantages of the invention will become apparent from the following detailed description when taken in conjunction with the accompanying drawings, which are a part of this disclosure and which illustrate, by way of example, principles of the invention.

### BRIEF DESCRIPTION OF DRAWINGS

Further aspects of the present invention will become apparent from the following description which is given by way of example only and with reference to the accompanying drawings in which:

FIG. 1 is an exploded view showing components of a system according to an aspect of the invention;

FIG. 2 is a view showing components of a splint according to an aspect of the invention relative to a patient's arm;

FIG. 3 is a bottom view of a splint according to an aspect of the invention;

FIG. 4 is a perspective view of the splint of FIG. 3;

FIG. 5 is a perspective view of a fastener tool according to an aspect of the invention;

FIGS. 6 to 10 are images showing steps in a method of using a splint according to an aspect of the invention;

FIG. 11 is a first view of a restraint portion according to an aspect of the invention;

FIG. 12 is an end on view of the restraint portion of FIG. 11;

FIG. 13 is a first view of a further restraint portion according to an aspect of the invention;

FIG. 14 is an end on view of the restraint portion of FIG. 13;

FIG. 15 is a bottom view of a splint according to an aspect of the present invention;

FIG. 16 is a top view of the splint of FIG. 15;

FIG. 17 is a perspective view of the splint of FIGS. 15 and 16;

FIG. 18 is a first view of a restraint portion of the splint of FIGS. 15 to 17 fitted to a patient's arm;

FIG. 19 is a perspective view of FIG. 18;

FIG. 20 is a top view of FIG. 18;

FIG. 21 is a side view of FIG. 18;

FIG. 22 is an exploded view of the splint of FIGS. 15 to 21;

FIG. 23 is a bottom view of a restraint portion of the splint of FIGS. 15 to 22;

FIG. 24 is an end on view of the restraint portion of FIG. 23;

FIG. 25 is a first perspective view of the restraint portion of FIG. 23;

FIG. 26 is a second perspective view of the restraint portion of FIG. 23;

FIG. 27 is a bottom view of a first alternate restraint portion according to an embodiment of the invention;

FIG. 28 is an end on view of the first alternate restraint portion of FIG. 27;

FIG. 29 is a first perspective view of the first alternate restraint portion of FIG. 27;

FIG. 30 is a second perspective view of the first alternate restraint portion of FIG. 27;

FIG. 31 is a bottom view of a second alternate restraint portion according to an embodiment of the invention;

FIG. 32 is an end on view of the second alternate restraint portion of FIG. 31;

FIG. 33 is a first perspective view of the second alternate restraint portion of FIG. 31;

FIG. 34 is a second perspective view of the first alternate restraint portion of FIG. 31;

FIG. 35 is a first close-up view of a sleeve and arm according to an aspect of the invention;

FIG. 36 is a second close-up view of the sleeve and arm of FIG. 35;

FIG. 37 is a third close-up view of the sleeve and arm of FIGS. 35 and 36;

FIG. 38 is a bottom view of an arm and a second restraint portion according to an aspect of the invention;

FIG. 39A is a side view of a first restraint portion according to an aspect of the invention;

FIG. 39B is a first view of Section A-A shown in FIG. 39A;

FIG. 39C is a second view of Section A-A shown in FIG. 39A;

FIG. 40A is a first view of a sizing jig according to an aspect of the invention;

FIG. 40B is a second view of a sizing jig according to an aspect of the invention;

FIG. 40C is a third view of a sizing jig according to an aspect of the invention; and

FIG. 41 is representative view showing use of sizing jog according to an aspect of the invention.

### BEST MODES FOR CARRYING OUT THE INVENTION

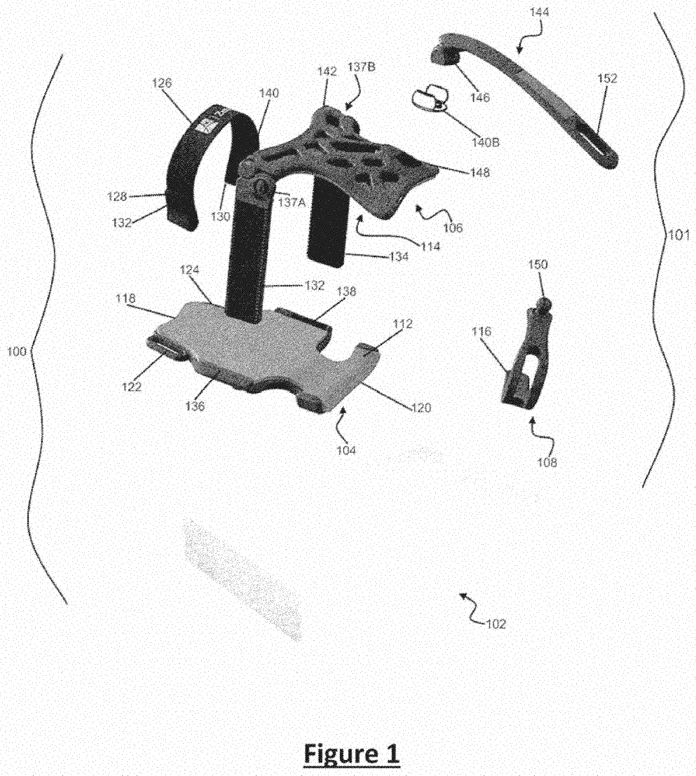

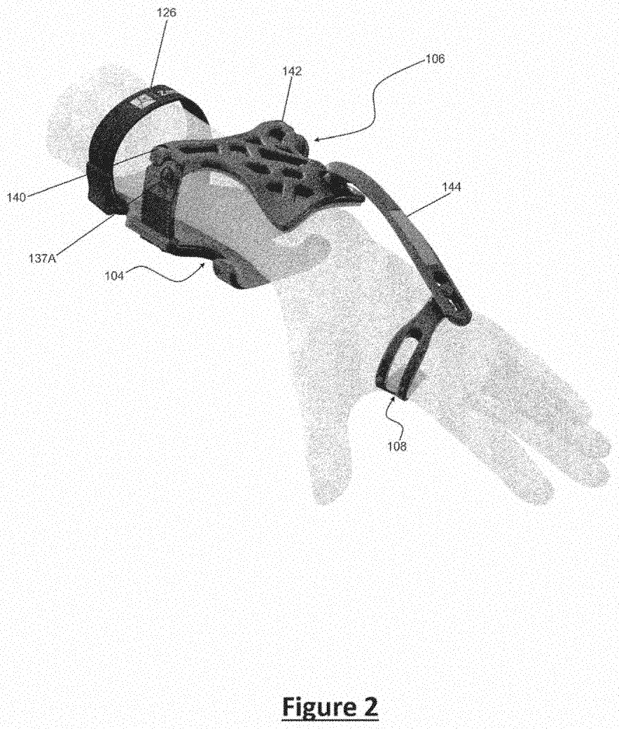

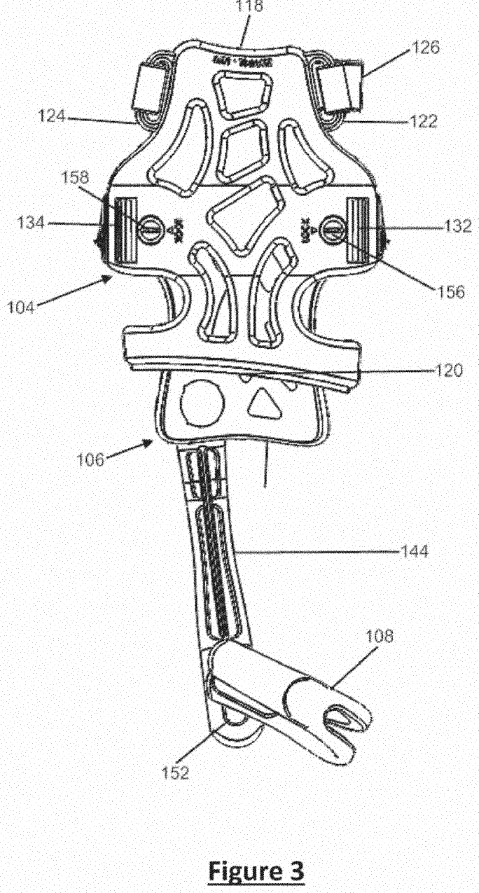

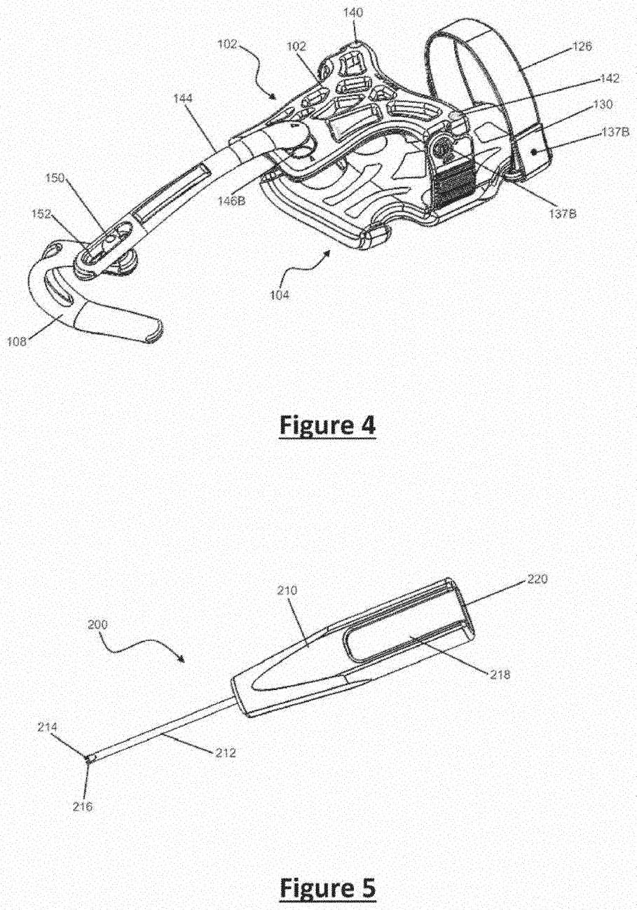

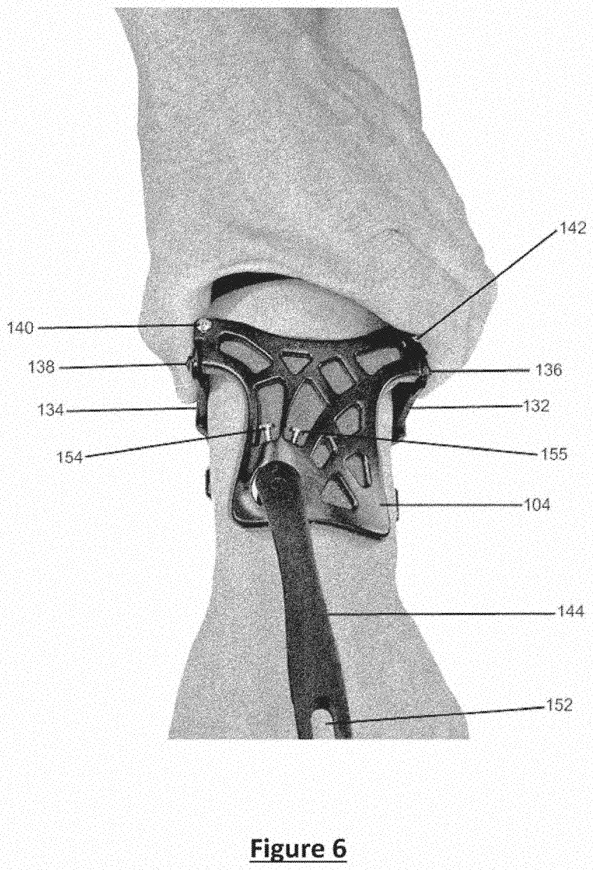

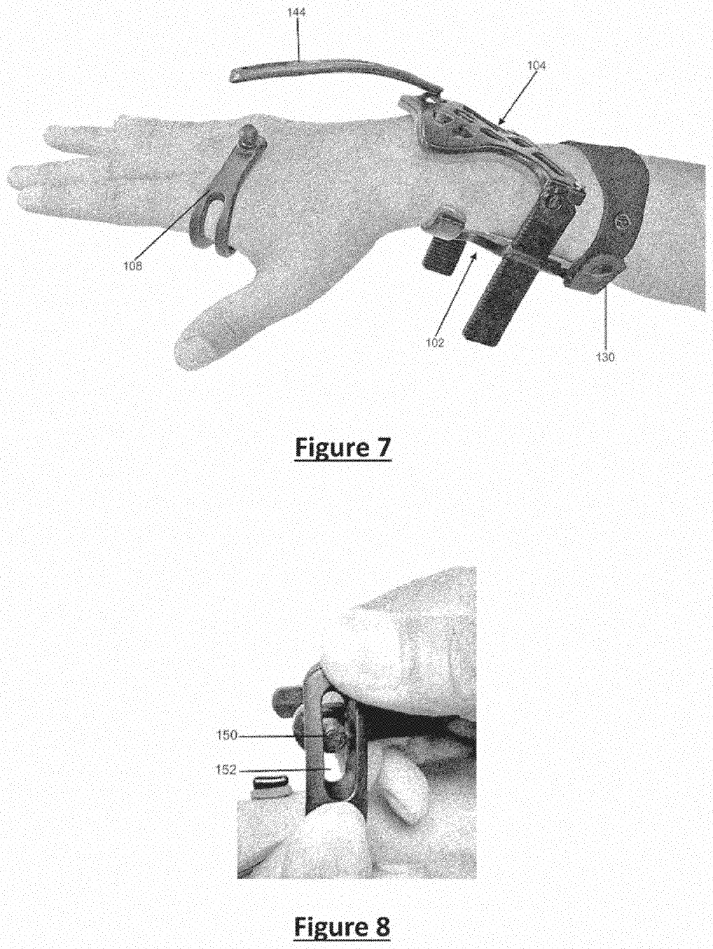

Referring first to FIGS. 1 to 9, there is provided a system (100), for use in treating a fracture e.g. a distal radius fracture in a human patient.

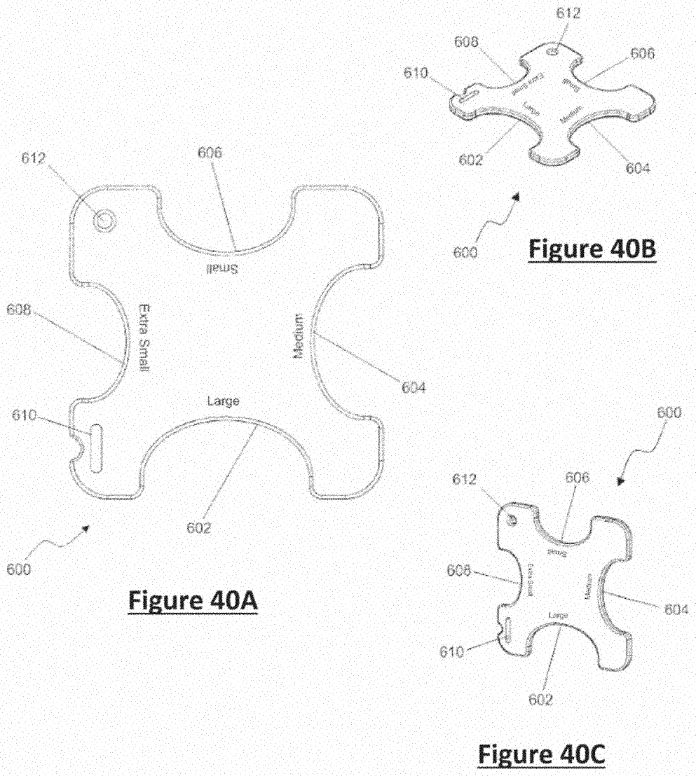

In the embodiment of FIGS. 1 to 9, the system (100) is sold as a kit set of parts comprising at least a splint (101) and a container (102). The system (100) may also include a sizing jig (600) as illustrated in FIGS. 40A-C.

The splint (101) includes a first restraint portion (104), a second restraint portion (106), and also optionally a third restraint portion (108). The splint (101) may be a splint as substantially described in PCT Application No. NZ2014/000117, and that document is incorporated herein in its entirety by reference. Additional features of the splint (101) and the improvements it provides over the splint described in PCT Application No. NZ2014/000117 should become clearer from the following discussion.

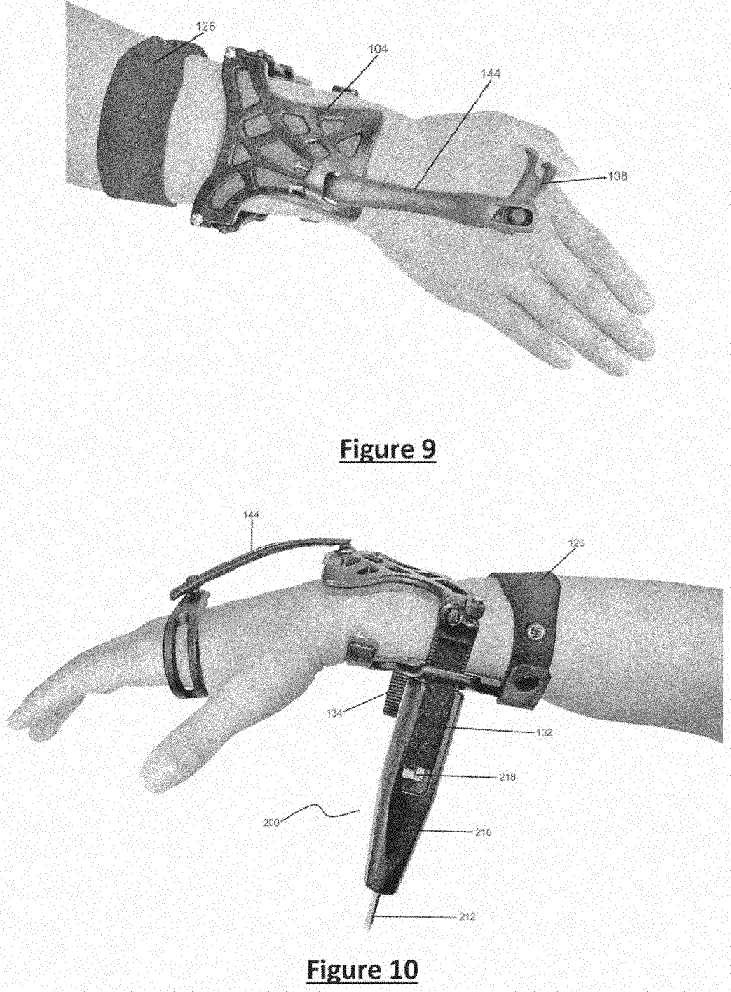

The system (100) may also include a tool (200) as is illustrated in FIG. 9, which is configured to facilitate fitting of the splint (101) to a patient (as will be discussed in more detail below).

In addition, the kit set of parts may include instructions (110) on how to use the splint (101).

The first restraint portion (104), second restraint portion (106), and third restraint portion are substantially rigid, and they can be made from materials such as acetal (POM), nylon, metal, other types of plastic materials, or combinations thereof.

The restraint portions (104 and 106) may each have a liner indicated as (112 and 114) respectively in the Figures. The restraint portion (108) may also have a liner (not shown in the Figures).

The liners are preferably soft, biocompatible materials. These may improve patient comfort or otherwise make them more suitable for use.

In a preferred embodiment, at least one of the liners (112 114) are structured and/or configured to provide a wicking effect. That is, the liners (112, 114) in-use draw moisture away from a patient's skin and into the liner(s). However, the moisture which may be absorbed by the liner(s) is not allowed to flow out of the liner onto the patient's skin.

This may be achieved by selection of the material to have appropriate properties e.g. the liner(s) may be formed from a polyurethane foam. For instance, the foam may be an open-celled foam and have a pore size which creates or otherwise promotes a capillary action to achieve the desired wicking effect. Alternatively, the liner(s) may be made from a material such as Teflon™.

The first restraint portion (104) has a proximal end indicated generally by (118) and a distal end indicated generally by (120). A pair of loops (122, 124) are formed in the first restraint portion (104) at or towards the proximal end (118).

A strap (126) is provided to the first restraint portion (104). The strap (126) is configured to be threaded through the loops (122 and/or 124). In-use, the strap (126) may be positioned around a patient's arm.

In the embodiment shown in FIGS. 1 to 9, a first end (128) of the strap (126) is permanently secured to the loop (122), while a second end (130) is releasably secured to the loop (124). The permanent attachment of the first end (128) is achieved by rivet (132) which extends through the folded over strap (126).

The releasable attachment of the second end (130) may for example be achieved using press fit fasteners. For instance, a first type of fastener half is provided towards the second end (130) while at least one, and preferably two or more complementary fastener halves (not shown in the Figures) are provided towards the centre of the strap (126). This may provide releasable attachment of the second end (130) to the loop (124). In addition, it may also facilitate adjustment of the length of the strap (126).

It is also envisaged that the strap (126) could include clips (not shown) that in use engage corresponding structure on the first restraint portion (104) to releasably or permanently secure the two components together.

The second restraint portion (106) includes a first leg (132) and a second leg (134). The first leg (132) and the second leg (134) form part of a connection assembly which attaches the first restraint portion (104) and the second restraint portion (106) to each other.

Each of the first leg (132) and the second leg (134) include teeth (unnumbered) on their respective inner and outer surfaces.

The first restraint portion (104) includes a first connection slot (136) and a second connection slot (138). Each of the first connection slot (136) and the second connection slot (138) are configured to receive a respective leg (132, 134) of the first restraint portion (104).

The legs (132, 134) are each pivotally attached to the second restraint portion (106) by a respective hinge (137A, 137B).

The hinges (137A, 137B) enable the orientation of the second restraint portion (106) to be adjusted relative to the leg(s) (132, 134).

The legs (132, 134) are resilient and able to flex. Accordingly, the legs (132, 134) provide a spring force to urge the teeth into engagement with the sides of the first connection slot (136) and the second connection slot (138). This engagement provides a holding mechanism in which the spacing of the first restraint portion (104) and the second restraint portion (106) is fixed.

To assist in holding the first restraint portion and the second restraint portion at a fixed distance from each other, the legs (132, 134) as substantially incompressible along their length.

The splint (101) includes a locking mechanism configured to secure the second restraint portion (106) in a fixed orientation with respect to the first leg (132) and the second leg (134). In the embodiment shown in the Figures, the locking mechanism is provided by at least one screw such as the preferred form of a first screw (140) and a second screw (142).

The first screw and the second screw (140,142) each have non-conventional heads. For instance, as is illustrated, each screw (140, 142) includes a pair of circular apertures that are configured to receive corresponding prongs (214, 216) on the fastener tool (200).

The use of non-conventional fastener heads may reduce the ability of patients to adjust the splint (101) and therefore facilitate better treatment of a fracture.

The third restraint portion (108) is a substantially U shaped resilient component. It is configured to receive a side edge of the patient's hand.

An arm (144) is connected to the second restraint portion (106) by a ball (146) on the arm (144) which is positioned inside a socket (148) formed in the second restraint portion (106). It is also envisaged that the arm (144) could be formed integrally with the second restraint portion (106) and/or the third restraint portion (108).

In preferred embodiments, the ball (146) is non-circular, having a pair of parallel sides and a pair of curved sides.

In addition, a sleeve (146B) may be provided. The sleeve is shaped to abut the pair of parallel sides of the ball (146). The sleeve may also have a pair of curved outer surfaces with a radius substantially corresponding to a radius of the pair of curved side walls of the ball (146). Accordingly, in combination the sleeve and ball (146) provide a continuous curved surface corresponding to a surface of the socket (148).

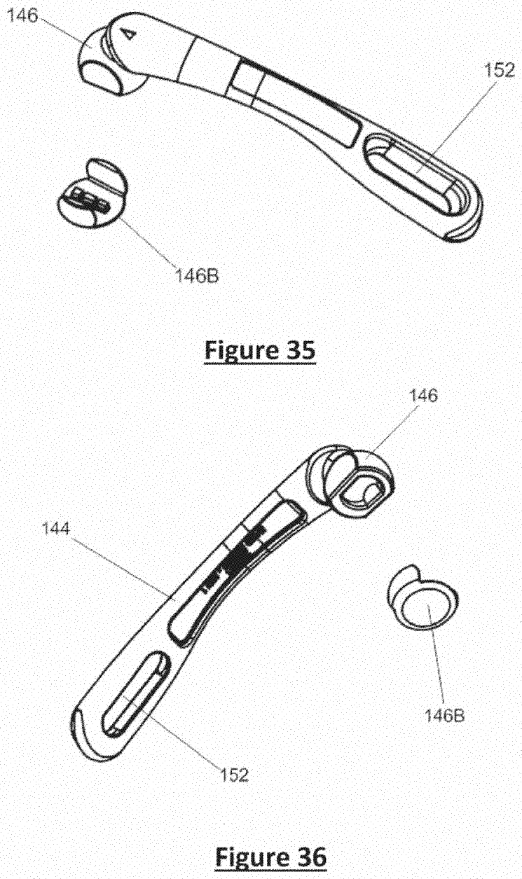

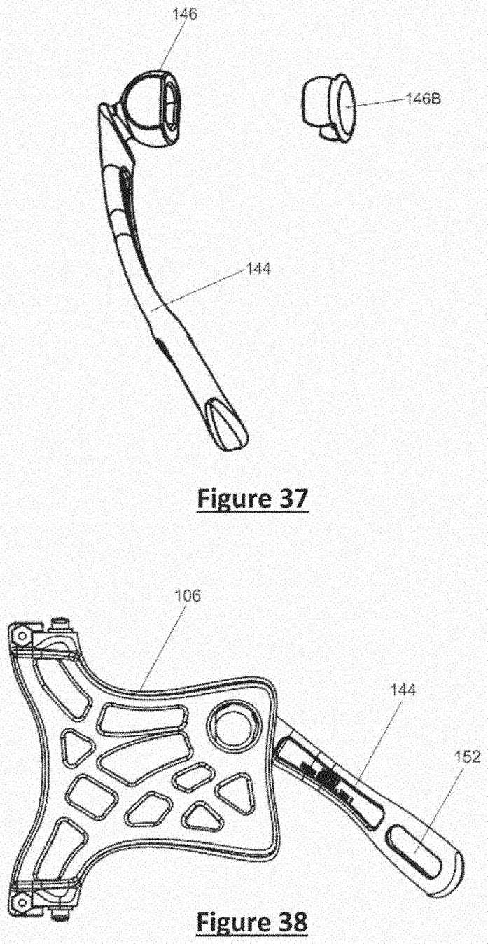

More detail of the sleeve (146B), ball (146) and arm (144) are shown in FIGS. 35 to 38.

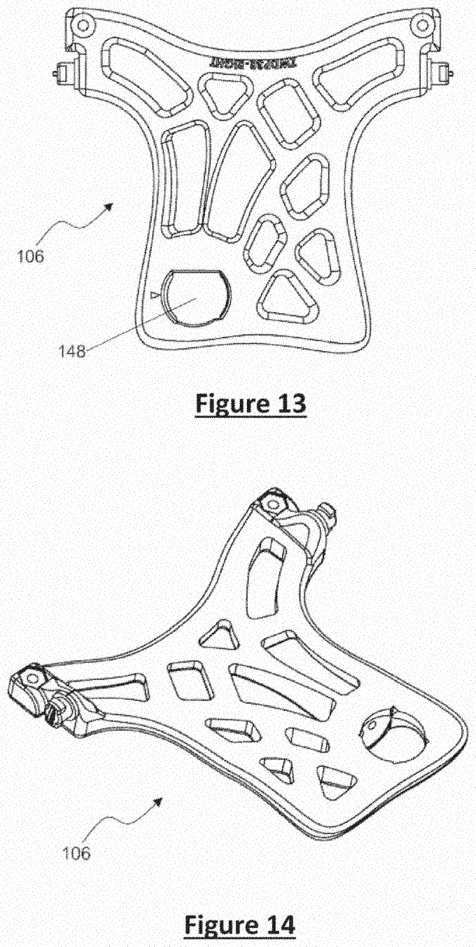

The socket (148) has an opening having a pair of parallel sides and a pair of curved sides, which generally correspond to the shape and dimensions of the ball (146). The parallel and curved sides of the opening in the socket (148) at best seen in FIG. 13.

To connect the arm (144) to the second restraint portion (106), the sleeve (146B) is first inserted into the socket (148) so that it aligns with the opening. The ball (146) is then inserted through the opening and into the sleeve (146B). The arm (144) can then be rotated, with the socket (146B) and ball (146) cooperating to provide a ball while a surface of the socket (148) provides an articulating surface for the ball. Rotation of the arm (144) causes the parallel sides of the ball (146) to be brought out of alignment with the parallel sides of the opening thereby providing resistance to the ball (146) being removed from the socket (148).

The third restraint portion (108) is connected to the arm (144) by inserting a ball (150) on the third restraint portion (108) into a corresponding slot aperture (152) on the arm (144).

The arm (144) has a degree of flexibility along at least one plane. However, the arm (144) is substantially incompressible. The flexibility allows a degree of movement in or about the patient's wrist joint e.g. the wrist may move towards extension and/or flexion to apply pressure into the arm (144). This may be beneficial for patient comfort or to promote healing of the fracture.

The splint (101) may also include a locking mechanism to prevent the separation of the first restraint portion (104) and the second restraint portion (106) being changed. In preferred forms, this locking mechanism is in addition to the interaction of the teeth with the connection slots (136, 138). For instance, the locking mechanism may be provided by one or more locking elements (156, 158). In the embodiment of FIGS. 1 to 9, the locking elements (156, 158) are rotating elements which can be moved between engaged and disengaged positions. In the engaged position, the locking element(s) (156, 158) engage the teeth to prevent the legs being moved through the connection slots (136, 138). In the disengaged position, the locking element(s) (156 and/or 158) do not prevent movement of the legs (132, 134) through the connection slots (136, 138).

The locking mechanism may include visual indications to indicate when the locking elements (156, 158) are in the engaged and disengaged positions. For instance, the visual indications may be a provided by pair of symbols e.g. triangles (unlabelled in the Figures) which align to indicate that the locking elements are in the engaged position.

The splint (101) includes a locking mechanism to selectively prevent movement of the ball (146) relative to the second restraint portion (106). In the illustrated embodiment, the locking mechanism is provided by at least one, and preferably a pair of, screws (154, 155). The screws (154, 155) preferably each have a non-conventional head which can be engaged by an engagement portion of a fastener tool (as is discussed in more detail below).

Referring now to FIG. 9 which shows the fastener tool (200) according to an aspect of the present invention. The fastener tool includes a body (210) and an extension portion (212). An engagement portion (indicated generally by 213) is provided to an end of the extension portion (212) which is distal to the body (210). The engagement portion is configured to engage with a locking mechanism of the splint (101) to facilitate moving the locking mechanism between the locked and release position (as will be discussed in more detail below).

In the illustrated embodiment, the engagement portion (213) is provided by a pair of prongs (214, 216).

The prongs (214, 216) are configured to engage corresponding apertures in screws forming part of locking mechanism(s) of a splint according to an aspect of the invention. When the prongs (214, 216) are engaged in the screws, the fastener tool can be rotated to also rotate the screw(s).

The fastener tool also includes a channel (218). The channel is shaped and configured to receive an end of the first leg and/or the second leg (132, 134). An edge (220) of the body (200) defining an outer end of the channel (218) provides a surface against which the legs may be flexed to in-use snap the legs off. Accordingly, the fastener tool (200), through use of the channel (218), and the shape/configuration of the body (210) facilitates adjusting the length of the first leg (132) and/or the second leg (134).

Referring now to FIGS. 40A-40C showing views of a sizing jig (600) according to an aspect of the present invention.

The sizing jig (600) is a low profile, slim component. It can therefore form part of the system (100) and fit in container without significantly increasing the bulk of the system (100). Alternatively, and/or in addition, the sizing jig (600) can be provided to medical practitioners and clinicians as a separate component rather than as part of the system (100), and its low profile, slim construction can allow it to be easily stored.

The sizing jig (600) has at least one channel configured to receive the patient's arm. In the preferred embodiment illustrated in FIGS. 40A-C, the sizing jig (600) has four channels, which are indicated as (602, 604, 606, 608).

Each channel (602, 604, 606, 608) corresponds to a suitable size of splint (100). As illustrated, each channel (602, 604, 606, 608) has markings indicating the size of splint to which it corresponds e.g. large, medium, small, and extra-small.

Alternatively, it is envisaged that the sizing jig (600) may have a single channel. In this embodiment, multiple sizing jigs may be provided or used, each of which corresponds to a different size of split (100).

The sizing jig (300) may also be provided with a slot (310). The slot (310) can be used to “snap” the legs of the splint to a desired length (as discussed in more detail below).

The sizing jig (600) may also be provided with an aperture (612). The aperture (612) may facilitate storing the sizing jig (600) e.g. it can be hung on a hook such as those used on plaster trolleys in medical centres.

Other aspects of the system (100), splint (101) and sizing jig (600) should become clearer from the following discussion describing its method of use.

### Method of Use

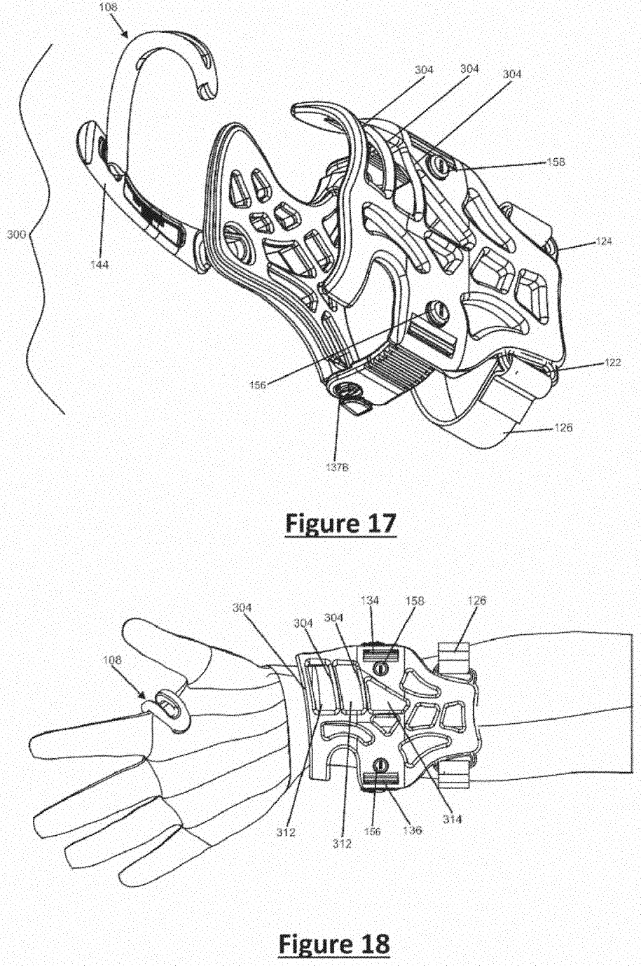

Referring now to FIGS. 10 to 20, a method of using of a splint (101) according to the present invention will now be described. It should be appreciated that the following steps may be performed in any order according to a patient's needs and/or a medical practitioner's preference.

If a patient suffers a facture e.g. a distal radial fracture in the patient's arm (700) then a splint (101) according to one aspect of the invention may be utlised for fracture reduction.

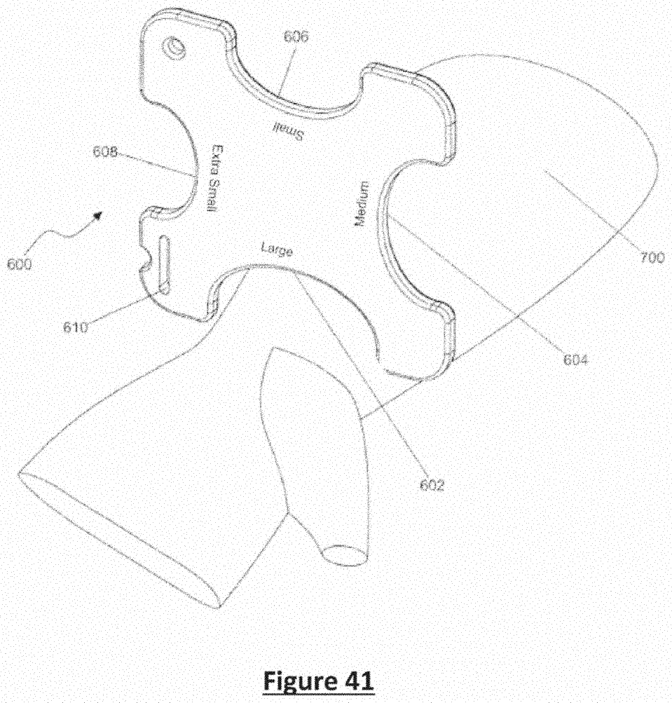

The sizing jig (600) is used to select the size of the splint (101) required for use in treating the patient's (700). As shown in FIG. 41, the sizing jig (600) is positioned against a surface of the patient's arm (700), e.g. the volar surface. To identify the correct location, a spacer such as a person's fingers may be positioned against a visible reference on the patient's arm (700). A preferred visible reference point is the patient's wrist crease, but alternatives includes bony protuberances in or around the wrist joint.

The sizing jig (600) is configured to measure the width of the patient's arm at approximately 8 cm from the wrist crease. The inventors have found that this may be particularly useful as it can assist to ensure that the legs of the splint (101) are spaced from the patient's arm and do not pinch the skin. In these embodiments, the person may use four fingers to measure the distance from the wrist crease to the correct location at which the sizing jig (600) is to be used.

It is also envisaged that the sizing jig may be provided with a spacer such as a ledge/step (not illustrated) in the Figures permanently or releasably attached to the sizing jig (600).

The channel (602) of the sizing jig (600) receives the patient's arm (500) and a person e.g. clinician can read that the size “large” splint is required to treat the patient. Alternatively, if the channel (602) is too large for the patient's arm (500), the person may rotate the sizing jig (600) to assess whether the channels (604, 606, 608) better fit the patient's arm (700). If one of the channels (604, 606, 608) is a better fit, then the size indication is noted by the person, and the appropriate splint (101) is selected for use in treating the person.

The first restraint portion (104) is positioned to abut the volar/anterior of the patient's arm as shown in FIG. 10. As shown in the Figures, the distal end of the first restraint portion is approximately two fingers away from the patient's wrist crease.

The second restraint portion (106) is positioned over the dorsal/posterior surface of the patient's forearm. The first leg and second leg (134) are aligned with the first connection slot (136) and the second connection slot (138) respectively.

The first leg and the second leg (132, 134) are inserted into and through the first connection slot and the second connection slot.

This brings the second restraint portion (106) into contact with the dorsal/posterior surface of the patient's arm.

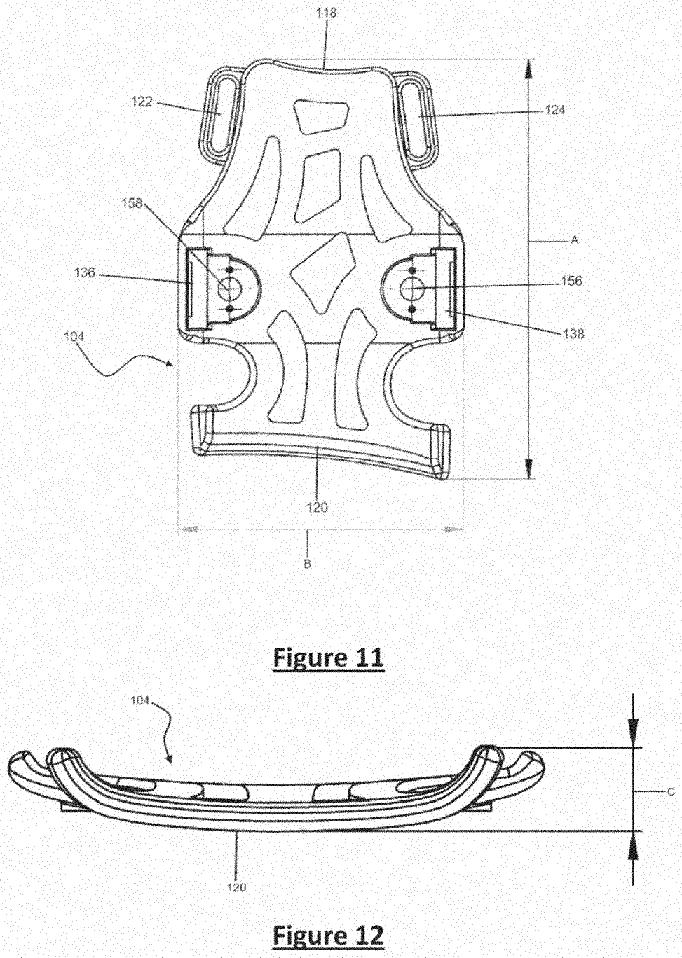

The strap (126) is wrapped around the patient's arm, and the end (130) is inserted through the loop (124). The fastener half is caused to engage one of the complementary fastener halves on the strap (126) to releasably secure the strap around the patient's arm and within the loop (124). This is the arrangement shown in FIG. 11.

In the alternative to the above, the splint (101) may be partially or completely assembled. The patient's arm can then be inserted into the gap between the first restraint portion (104) and the second restraint portion (106).

If the spacing of the first restraint portion (104) and the second restraint portion (106) needs to be adjusted, then the legs can be pinched together as shown in FIG. 12. This brings the ratchet teeth out of engagement with the connection slots and enables the restraint portions (104, 106) to be moved towards and/or away from each other.

The locking elements may be engaged by the prongs (214, 216) and rotated to move these into the engaged position. This brings the visual symbols into alignment. The locking elements prevent the legs (132, 134) moving in the connection slots (136, 138).

The second restraint portion (106) may be pivoted with respect to the first leg (132) and the second leg (134) by rotating the second restraint portion (106) about the hinges (137A, 137B). This applies pressure to the dorsal/posterior surface of the patient's arm to change the orientation of the bone fragments relative to each other to partially or completely reduce the fracture. Contact between the first restraint portion (104) and the volar/anterior surface of the patient's arm provides a reactionary force to pressure applied by the second restraint portion (106).

The open construction of the first restraint portion (104) and the second restraint portion (106) enables a medical practitioner to sight bone protrusions in the patient's forearm and therefore it may assist to achieve the correct or desired alignment of the bone fragments. This can be distinguished from prior art methods for fracture reduction and treatment such as plaster casts in which the protrusions are obscured. Accordingly, a splint according to the present invention may provide a better solution for fracture reduction.

If required, lateral pressure may be applied to at least one of, and preferably both of, the first leg (132) and the second leg (134). The pressure disengages the ratchet teeth from the edges of the respective connection slots (136, 138). The legs (132, 134) can be partially or completely withdrawn from the connection slots (136, 138). Accordingly, the first restraint portion (104) and the second restraint portion (106) may be moved apart from, or completely separated from, each other. This may be beneficial to adjust the separation of the restraint portions (104, 106) with respect to each other and the pressure which they apply to the patient's arm. In addition, this may enable further adjustment of the orientation of the restraint portions (104, 106), and therefore better alignment of the bone fragments.

The fastener tool (200) can be used to rotate the screws (140, 142) to lock the orientation of the second restraint portion (106) relative to the first restraint portion (106), as is shown in FIG. 14.

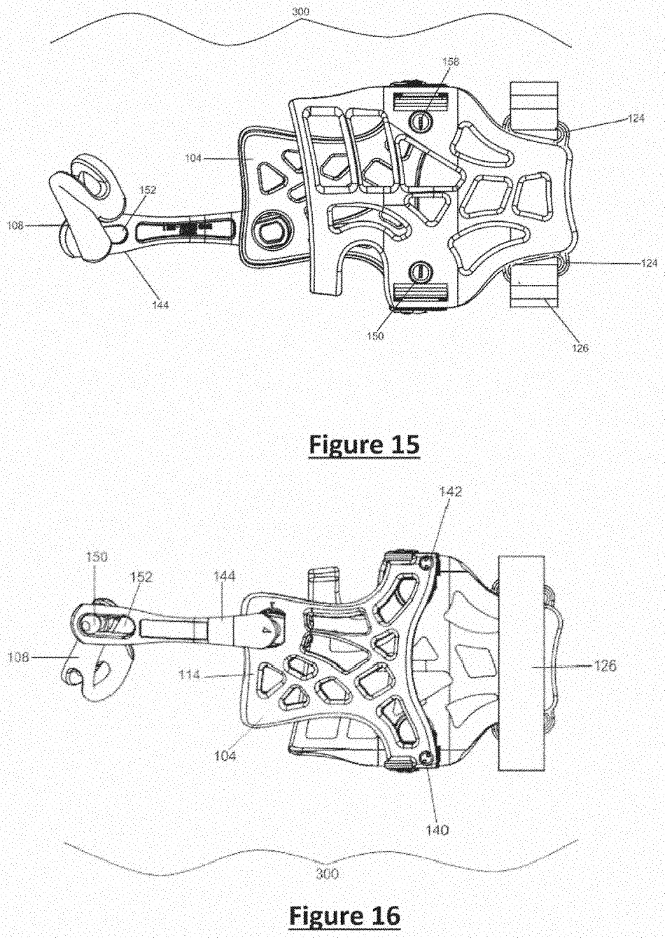

The third restraint portion (108) is positioned to receive a side edge of the patient's hand, as shown in FIG. 15.

The arm (144) is attached to the third restraint portion (106) by pushing the ball (150) into the slot aperture (152), as shown in FIG. 16.

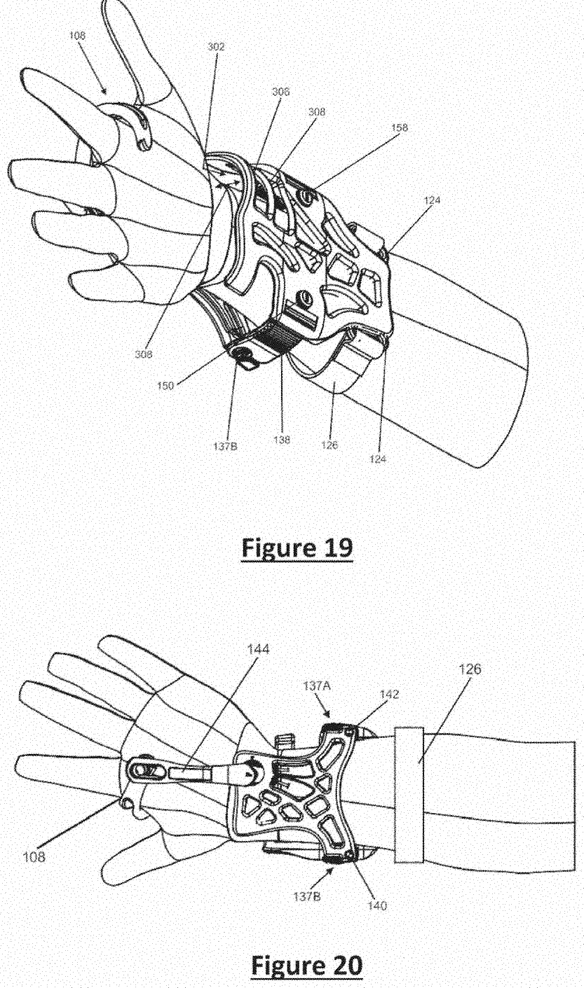

The orientation of the arm (144) relative to the second restraint portion (106) is fixed using the locking mechanism. To do so, the prongs (214, 216) engage the apertures in the screws and the fastener tool (200) is rotated to cause the screws to clamp onto the ball (146). The splint is now fitted to the patient and the bone fragments have been orientated to reduce the fracture as is shown in FIG. 19.

The fastener tool (200) is positioned so that an end of the first leg (132) is adjacent to the channel (210) and the end is then inserted into the channel 210. The fastener tool is then moved to snap the end of the leg off from the remainder of the (132) leg. This process is then repeated for the second leg (134).

Alternatively, the sizing jig (600) can be used to snap off the ends of the legs (132, 134), but inserting the legs (132, 134) into the slot (610) in turn, and moving the sizing jig (600) and leg(s) (132, 134) relative to each other.

### Fracture Support Surface

The splint (101) according to the invention is structured and/or configured to contact the patient's forearm arm in use over a large area e.g. it may contact a substantial part of, or the entirety of, the forearm region. This may be beneficial in designing a splint which can better adapt to anatomical variations across the population. However, this should not be seen as requiring that the splint contact all surfaces of the forearm. Instead, the splint can contact the forearm at two or more spaced apart points, leaving at least some portion of the arm surface(s) between the spaced apart points open or uncontacted.

The distal end of the patient's forearm is primarily comprised of bones, tendons and ligaments whereas the proximal portion also includes significant muscle mass. There is significant variation in the dimensions of the muscle mass, and the size of the proximal portion of the forearm can have large variation across the population. Accordingly, substantially or completely avoiding the proximal portion of the forearm may facilitate design and use of a splint.

In a preferred embodiment, the first restraint portion (104) and the second restraint portion (106) provide a fracture support length of at least eight centimetres. That is, the total separation between the proximal end of the first restraint portion and the distant end of the second restraint portion (106) is at least eight centimetres.

The Applicant has identified that having a fracture support length of at least eight centimetres is particularly beneficial for facilitating fracture reduction and promoting healing of bone fractures.

The upper limit for the fracture support length could be the entire length of the patient's arm.

### Sizing

A system (100) according to the invention may be provided in one or more sizes e.g. an extra small, small, medium and large size. The respective sizes may differ from each other in one or more dimensions e.g. length, width, curve.

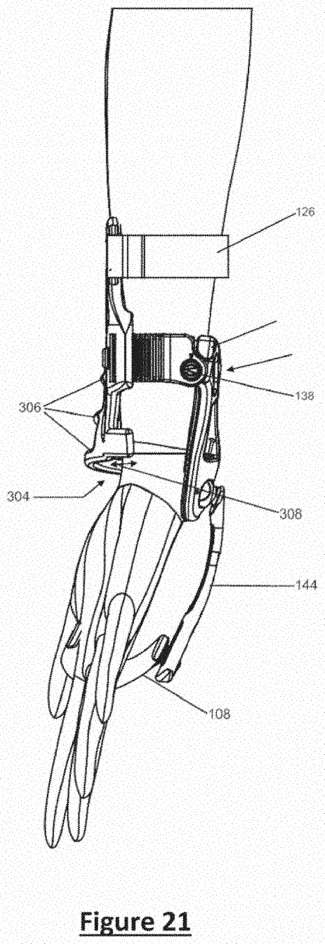

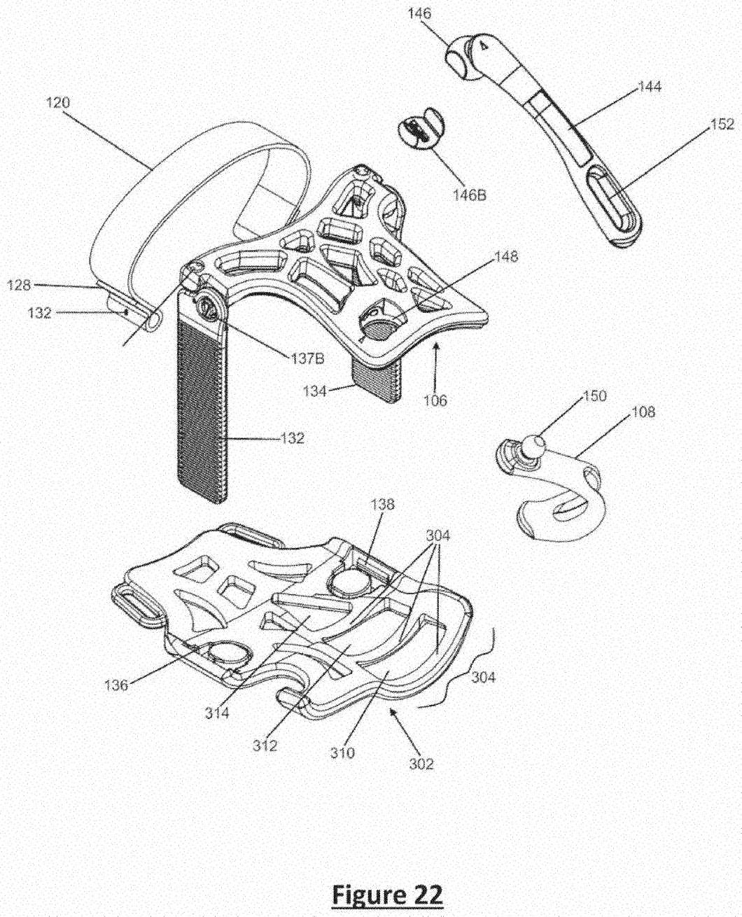

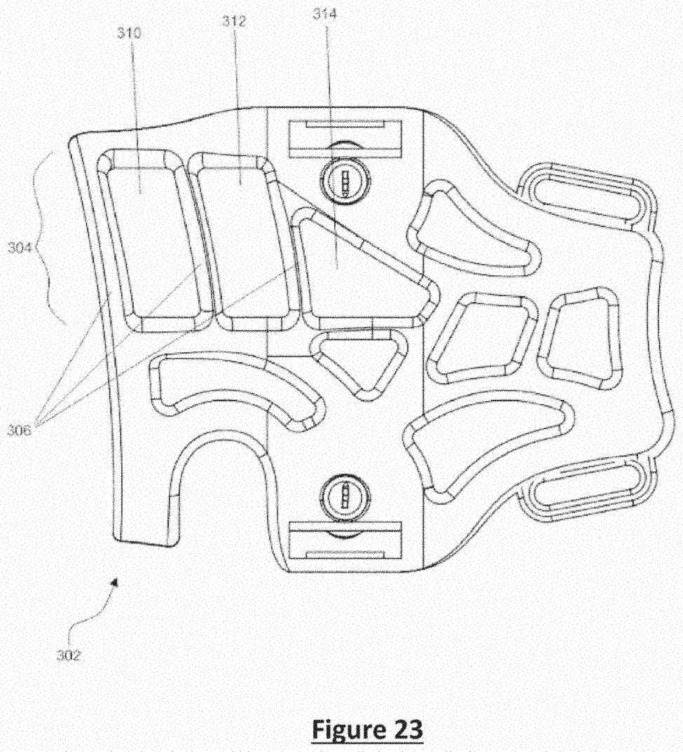

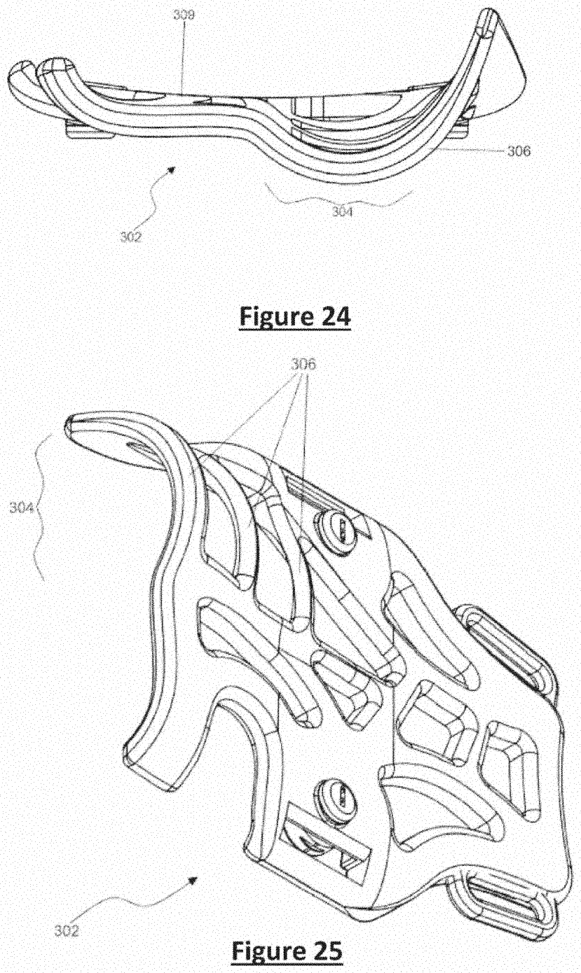

Referring now to FIGS. 21 and 22 which show a second restraint portion (146) and FIGS. 23 and 24 which show a first restraint portion (102) respectively. Table 1 below summarises preferred dimensions of the different sizes of the first restraint portion (104):

TABLE 1

| Dimension | Extra-small | | Small | | Medium | | Large | |
|---|---|---|---|---|---|---|---|---|
| A | 98 | mm | 109 | mm | 109 | mm | 109 | mm |
| B | 62.8 | mm | 73.5 | mm | 84.1 | mm | 94.8 | mm |
| C | 12 | mm | 15 | mm | 15 | mm | 15 | mm |

Table 2 below summarises preferred dimensions of the different sizes of the second restraint portion (106):

TABLE 2

| Dimension | Extra-small | Small | | Medium | Large |
|---|---|---|---|---|---|
| A | 74 mm | 79 | mmm | 79 mm | 84 mm |
| B | 65 mm | 75 | mm | 86 mm | 97 mm |
| C | 20 mm | 20 | mm | 19 mm | 19 mm |

However, it should be appreciated that the above dimensions are approximate only, and may vary such as by up to 10% of the noted values.

One or more of the sizes may overlap. For instance, the small and medium sizes may both be capable of use with a patient on the boundary of the respective sizes. This may improve fitting of the splint to patients, and assist with ensuring that the splints can be used by a portion of the population.

In addition, the liner(s) (112, 114) may assist to account for anatomical variations in patients' limbs. For instance, forming the liner(s) (112, 114) form a compressible material may allow the splint (101) to better adapt to the size of a user's limb. This can also enable given size of splint (101) to better adapt to patient's that may be on the boundary of a given size splint (101).

The preferred dimensions of the restraint portions according to the present invention may also be particularly useful in assisting the splint to fit to, and contact substantially only, the distal regions of a patient's arm. These regions of the arm have relatively less muscles/tendons and therefore the dimensions of that body part are more consistent across a population.

Furthermore, the ranges identified provide a useful demarcation between different patients.

Accordingly, the identified preferred dimensions may provide a splint which is better suited to treating a fracture.

As noted above, the system (100) according to the invention may also include a sizing jig (600) configured to assist in selecting the correct size of splint (101) for use with a given patient. A representative sizing jig (600) is shown in FIGS. 40A-C. To use the sizing jig (600), a person e.g. clinician places three fingers proximal to the ulnar prominence and the sizing jig (600) is positioned adjacent to the fingers. The orientation of the sizing jig (600) is adjusted to select the correct channel (602, 604, 606, 608) that best “fits” the patient's forearm. For instance, as shown in FIG. 41, the channel (602) corresponding to a size “large” splint receives the patient's arm (700). A recommended size for the patient can then be read from the sizing jig (600).

### Limb Specificity

A splint (101) according to the invention may be adapted for use with one of a patient's left hand or right hand. This can be achieved by the shape and design of at least one of, and preferably both of, the first restraint portion (104) and the second restraint portion (106). For instance, the shape of a first restraint portion (104) for use on a patient's left hand is a mirror image of the shape of a first restraint portion (104) for use on a patient's right hand. The same principle applies to second restraint portions (106) for use with the patient' right and left hands.

Accordingly, the invention may reside in provision of a splint (101) which is shaped and/or configured for use with one of a patient's left hand or right hand. A splint, (101) may be shaped and/or configured for use with a patient's right hand, while a different splint (101) may be configured for use with a patient's left hand.

Providing a splint (101) shaped and/or configured for use with one of a patient's left hand or right hand may facilitate better adaptability of the splint to the patient, improve comfort, facilitate improved alignment of bone fragments and improve usability.

In addition, the splint (101) may be structured and/or arranged to allow at least minimal movement at the wrist joint while preventing relative movement of bone fragments. Allowing movement at the joint may be beneficial as it assist in maintaining joint health while concurrently treating a bone fracture.

It should be understood that the amount of movement at the joint is variable. Enabling even minimal movement could be beneficial to patient health.

### Splint for Use Post-Surgical Fixation of Bone Fragments

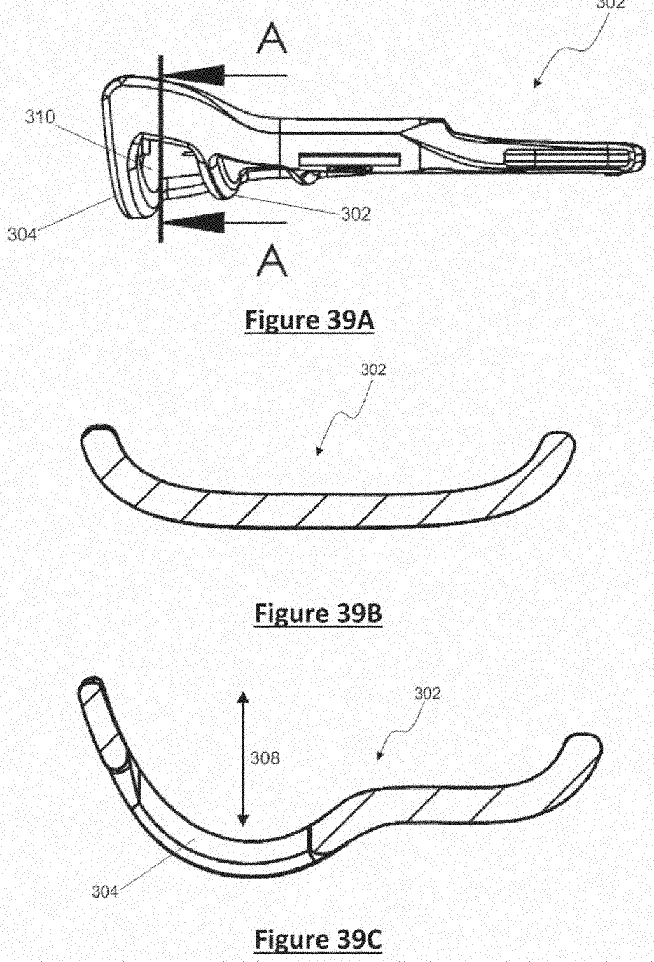

Referring now to FIGS. 15 to 26 and 39 A, B and C which illustrate a splint (300) configured for use after a fracture has been treated by surgical fixation.

The splint (300) has many components identical or substantially equivalent to the splint (101) discussed above with reference to FIGS. 1 to 9. Therefore, like references will be used to refer to like components. It should be appreciated that fitting and use of the splint (300) is substantially identical to the splint (100) discussed above. Therefore, only differences between the components of the splint and the consequences of these for use of the splint (300) will be discussed herein.

The splint (300) can be used to assist in maintaining bone fragments in proper alignment which have been fixed by surgical fixation, and to facilitate healing of these bone fragments.

Splint (300) has a first restraint portion (302) which is configured to in-use contact the volar surface of a patient's forearm.

The first restraint portion (302) differs to first restraint portion (104) in that it is designed to avoid contact with the region of the patient's arm in which an FCR incision is made. To do so, the first restraint portion (302) is structured to define a clearance zone, indicated as (304) in the Figures. The clearance zone (304) facilitates the first restraint portion (302) avoiding contact with the region of the volar surface of a patient's arm in which an FCR incision is made for surgical fixation of bone fragments, together with the surrounding area of the forearm.

In the embodiment illustrated in FIGS. 15 to 26, the clearance zone (304) is provided by bridge members (304), such as the preferred embodiment of three bridge members (304) as illustrated. However, it should be understood that one, two, four or more bridge members (304) could also be used.

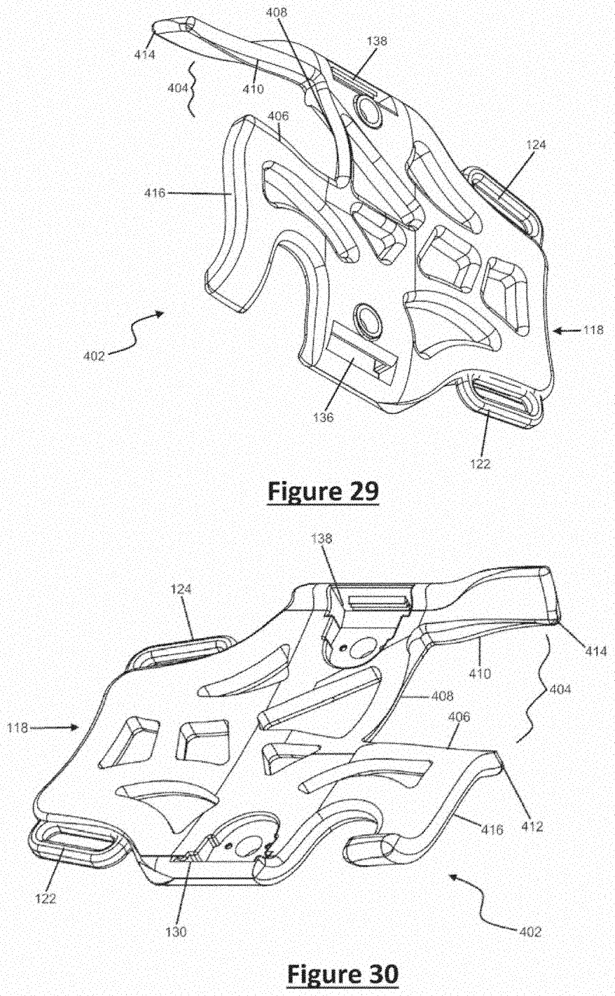

The bridge members (304) are curved and create a gap (308) between an inner surface (309) of first restraint portion (300) which lies over top of the surface of the patient's arm in which the FCR incision is made. The gap (308) is best seen in FIGS. 19 and 20, and FIG. 29C.

The first restraint portion (302) includes a distal edge, a proximal edge, a first lateral side edge, and a second lateral side edge. The bridges (304) are structured so that the distal side edge is able to provide pressure to the patient's forearm in an area of the patient's forearm distal to the location of the FCR incision.

In addition, the bridges (304) are structured to enable the first lateral side edge to contact the radial side of the patient's forearm, lateral to an FCR incision. The shape and configuration of the first lateral side edge is useful to ensure that the first restraint portion (300) has sufficient contact with the patient's forearm to ensure that it is stable and can apply the pressure required to keep bone fragments in a desired alignment.

The second lateral side edge of the first restraint portion (302) is identical to the equivalent lateral side edge of the first restraint portion (106) discussed above.

In addition, the first restraint portion (302) includes at least one window (310), and preferably a second window (312) and a third window (314). The windows (310, 312, 314) provide a line of sight to an FCR incision. Therefore, it is possible in inspect the FCR incision. The windows (310, 312, 314) and/or bridges (306) may also allow ventilation for the FCR incision and/or avoid pressure with the FCR incision. These foregoing features may be advantageous to facilitate healing of the FCR incision, improve patient comfort and treatment of a fracture.

### First Alternate Restraint Portion

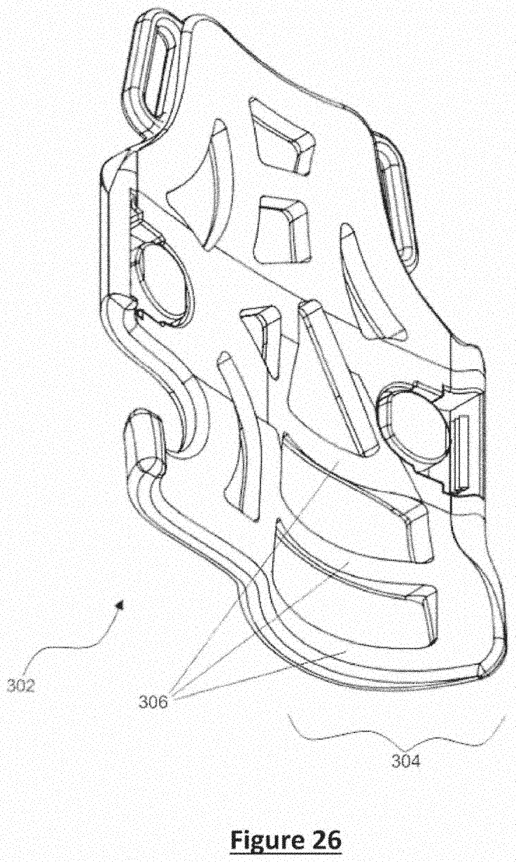

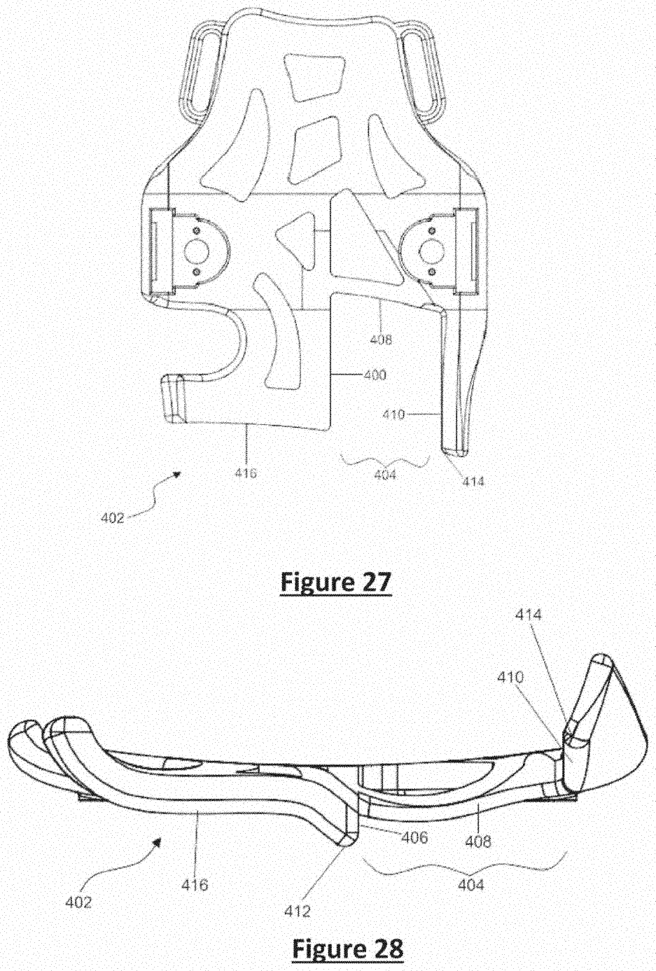

Referring now to FIGS. 27 to 30 which show an alternate restraint portion (402) according to an aspect of the invention.

The alternate restraint portion (402) is structured to define a clearance zone, indicated as (404) in the Figures. The clearance zone (404) is provided by a single window, which is defined by a first side edge (406), a second side edge (408) a third side edge (410) and an unbounded/open side edge (unnumbered).

The unbounded/open side edge (unnumbered) is defined by the gap between the distal end (412) of the first side edge (406) and the distal end (414) of the third side edge (410). The second side edge (408) is curved similarly to the bridge(s) of the first restraint portion (302) discussed above with reference to FIGS. 15 to 26.

The distal edge (416) of the alternate restraint portion (402) is able to provide pressure to the patient's forearm in a similar manner to the restraint portion (302) discussed above with reference to FIGS. 15 to 26. However, the provision of the clearance zone (404), ensures that a splint using the alternate restraint portion (402) does not contact the region of a patient's forearm in which an FCR incision is made, or the surrounding region of the forearm. In addition, the single window allows easy visual inspection of the FCR incision and ventilation of the wound to promote to healing.

### Second Alternate Restraint Portion

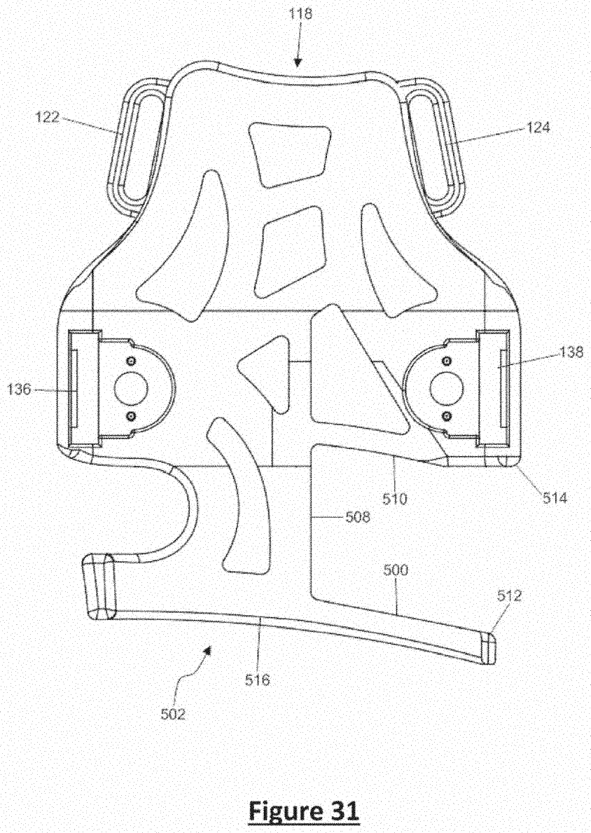

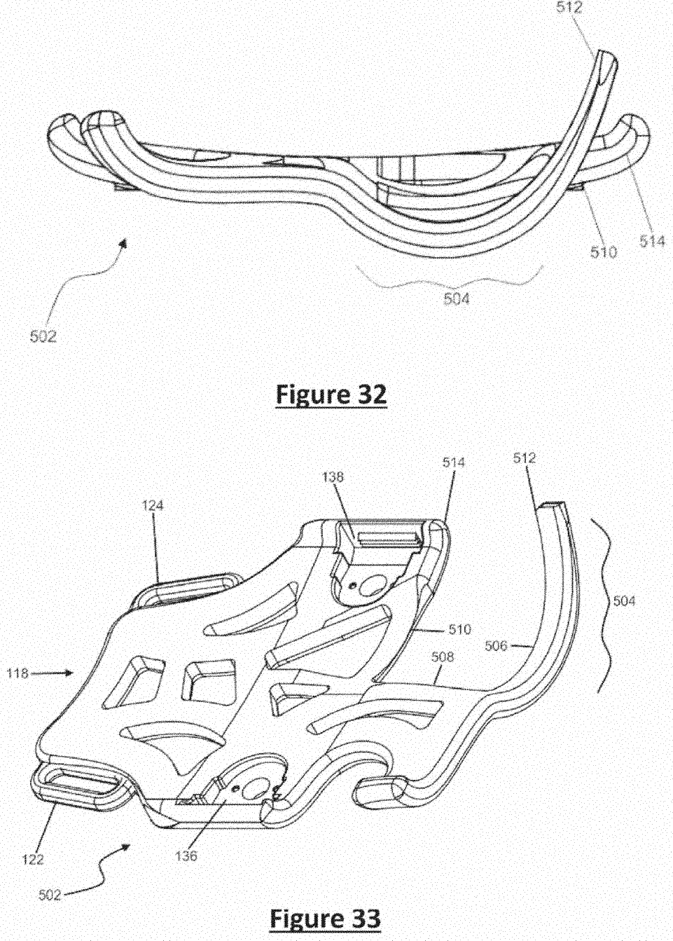

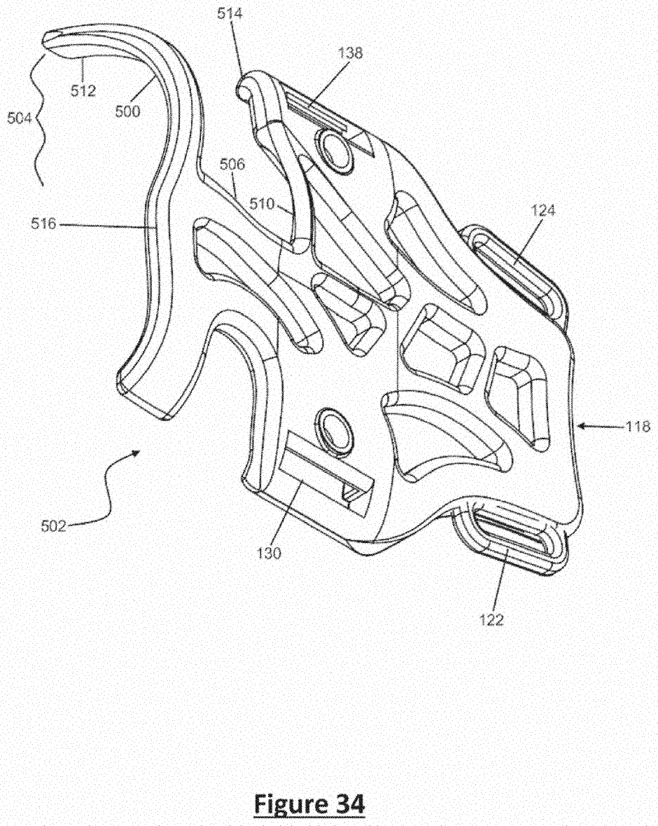

Referring now to FIGS. 31 to 34 which illustrate a second alternate restraint portion (502) according to an embodiment of the present invention.

The second alternate restraint portion (502) is structured to define a clearance zone (504) in the form of a single window. The single window is defined by a first side edge (506), a second side edge (508), a third side edge (510) and an unbounded/open side edge (unnumbered).

The unbounded/open side edge (unnumbered) is defined by the gap between a lateral end (512) of the first side edge (506) and a lateral end (514) of the third side edge (510).

The third side edge (510) is curved similarly to the bridge(s) (308, 310, 312) of the first restraint portion (302) discussed above with reference to FIGS. 15 to 26.

The clearance zone (404) ensures that the second alternate restraint portion (402) does not contact the region of the patient's forearm in which an FCR is made for surgical fixation of bone fragments, or the surrounding area.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment. Furthermore, each individual component of any given assembly, one or more portions of an individual component of any given assembly, and various combinations of components from one or more embodiments may include one or more ornamental design features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the present invention have been described by way of example only and it should be appreciated that modifications and additions may be made thereto without departing from the scope thereof.

1. A splint for use in reducing a fracture which produces a first bone fragment and a second bone fragment, wherein the splint comprises:
a first restraint portion configured to apply pressure to a volar surface of the patient's forearm and thereby apply pressure to at least one of the first bone fragment and the second bone fragment;
a second restraint portion configured to apply pressure to a dorsal surface of the patient's forearm and thereby apply pressure to at least one of the first bone fragment and the second bone fragment,
wherein the orientation of the first restraint portion and the second restraint portion can be adjusted with respect to each other to thereby align the first bone fragment and the second bone fragment in a desired orientation that substantially corresponds to an orientation for the first bone fragment and the second bone fragment in a non-fractured bone; and
a connection assembly between the first restraint portion and the second restraint portion, and
wherein the connection assembly can hold the first restraint portion and the second restraint portion substantially immovably with respect to each other to thereby hold the first bone fragment and the second bone fragment in the desired orientation.

2. The splint according to claim 1, wherein the connection assembly comprises at least one spacer element and a joint.

3. The splint according to claim 1, wherein the at least one spacer element comprises a first leg having a length.

4. The splint according to claim 3, wherein the at least one spacer element further comprises a second leg having a length.

5. The splint according to claim 4, wherein at least one of the first leg and the second leg is constructed or arranged to be snapped to adjust the length of the first leg or the second leg.

6. The splint according to claim 5, wherein at least one of the first leg and the second leg comprises one or more weakened regions to facilitate the first leg or the second leg being snapped.

7-42. (canceled)

43. The splint according to claim 3, wherein the first restraint portion comprises a channel configured to receive the first leg.

44. The splint according to claim 43, wherein the first leg comprises a plurality of teeth which engage the channel.

45. The splint according to claim 4, wherein the first restraint portion comprises a channel configured to receive the second leg.

46. The splint according to claim 45, wherein the second leg comprises a plurality of teeth to engage which engage the channel.

47. The splint according to claim 5, wherein at least one of the first leg and the second leg is resilient.

48. The splint according to claim 44, wherein at least one of the first leg and the second leg is structured or arranged to bias the teeth to engage the respective channel.

49. (canceled)

50. The splint according to claim 1, wherein the splint is provided in an extra-small, small, medium, or large size.

51. The splint according to claim 50, wherein the sizes of the first restraint portion are substantially:

| Dimension | Extra-small | | Small | | Medium | | Large | |
|---|---|---|---|---|---|---|---|---|
| A | 98 | mm | 109 | mm | 109 | mm | 109 | mm |
| B | 62.8 | mm | 73.5 | mm | 84.1 | mm | 94.8 | mm |
| C | 12 | mm | 15 | mm | 15 | mm | 15 | mm |

52. The splint according to claim 50, wherein the sizes of the second restraint portion are substantially:

| Dimension | Extra-small | Small | Medium | Large |
|---|---|---|---|---|
| A | 74 mm | 79 mm | 79 mm | 84 mm |
| B | 65 mm | 75 mm | 86 mm | 97 mm |
| C | 20 mm | 20 mm | 19 mm | 19 mm |

53. The splint according to claim 1, further comprising a tool configured to facilitate fitting of the splint to a patient.

54-67. (canceled)

68. The splint according to claim 53, wherein the first restraint portion is structured to define a clearance zone which is configured to ensure that the first restraint portion does not contact an area of the patient's forearm in which an FCR incision is made.

69-72. (canceled)
